(19) United States
(12) Patent Application Publication
(10) Pub. No.: US 2011/0166434 A1
(43) Pub. Date: Jul. 7, 2011

(54) SYSTEM FOR SENSING ELECTROPHYSIOLOGICAL SIGNALS

(76) Inventor: Gaetano Gargiulo, New South Wales (AU)

(21) Appl. No.: 13/002,904

(22) PCT Filed: Jul. 6, 2009
(86) PCT No.: PCT/AU2009/000873

§ 371 Date: Feb. 16, 2011

(30) Foreign Application Priority Data
Jul. 7, 2008 (AU) 2008903464

(51) Int. Cl. A61B 5/04; H01B 5/00; H02G 3/00

(52) U.S. Cl. 600/372; 174/126.1; 174/70 R

(57) ABSTRACT

A system (10) for sensing electrophysiological signals includes a plurality of shielded electrodes (12). The electrodes (12) are connected to an amplifier (18) for amplifying signals received from each of the electrodes (12), the amplifier having a shielding input and the shielding of the electrodes (12) being connected to the shielded input of the amplifier (18). A power supply (22) powers the amplifier (18), a reference voltage derived from the power supply (22) being used as a reference voltage for the electrodes (12).

### CROSS-REFERENCE TO RELATED APPLICATIONS

The present application claims priority from Australian Provisional Patent Application No 2008903464 filed on 7 Jul. 2008, the contents of which are incorporated herein by reference.

### FIELD OF THE INVENTION

This invention relates, generally, to the sensing of physiological signals and, more particularly, to a system for sensing electrophysiological signals and to an electrode for use with the system.

### BACKGROUND TO THE INVENTION

In the sensing of physiological signals, such as in ECGs, EEGs or EMGs, use is made of a plurality of electrodes which need to be attached to the skin of a subject being investigated. The accuracy of any resulting test is dependent on the quality of the contact made between the skin of the subject and the electrode.

To enhance the skin/electrode contact, use is generally made of a conductive gel or paste which is applied between the electrode and the skin. It is also often necessary to prepare the site to which the electrode is to be attached to enhance skin/electrode contact. For example, in hirsute individuals, it may be necessary to shave the site so that hair follicles do not adversely affect skin/electrode contact.

The use of a paste has problems in that there can be leakage of signals through the paste between adjacent electrodes. Also, in wet or humid environments, there is a risk of the electrode's contact with the skin being reduced. In addition, should the paste dry out, noise artefacts in the signals increase.

Further, in some cases, such as in EEG recordings, each electrode needs to have an amplifier associated with it and also use is made of a ground and/or reference electrode to enable measurements to be taken. The weight and physical size of such an arrangement reduces the number of electrodes available for a multi-channel recording.

There is a further danger of having printed circuit boards containing powered components in contact with or close to a patient's skin.

Still further, there may be instances where it is not possible to place the electrodes in direct contact with the skin of the individual. There may be instances where it is desired or necessary to have the electrodes in proximity to, but not in contact with, the skin of the individual.

### SUMMARY OF THE INVENTION

According to a first aspect of the invention, there is provided an electrode for a system for sensing electrophysiological signals, the electrode comprising

a carrier;

a conductive element mounted on the carrier; and

a shielding element carried by the carrier and arranged in spaced relationship relative to the conductive element.

The conductive element may have an impedance which is greater than 100Ω.

The carrier may be of an insulating material. The insulating material may be a non-conducting elastomeric material. The elastomeric material may be a natural or a synthetic rubber material.

The conductive element may be at least one of a metal element which is, preferably, biocompatible and a conductive elastomer. For example, the metal may be selected from the group consisting of silver, gold, plated silver or gold and medical grade stainless steel. The conductive elastomer may be a silicone-based elastomer containing conductive material such as a carbon material or a platinum material in granular form. The conductive material may be mixed in the silicone in the desired quantity.

The conductive element may adopt various shapes. In an embodiment, the conductive element may be substantially planar and may be polygonal or circular. The conductive element may be in the form of a pad or a grid-like lattice structure. In another embodiment, the conductive element may be three dimensional and may be substantially spherical.

The shielding element may have a shape complementary to its associated conductive element. The shielding element may be maintained by the carrier in spaced, parallel relationship to at least a part of a periphery or a surface of the conductive element.

The shielding element may be a conductive plate, for example, of an aluminium or a copper material shaped to complement the periphery or surface of the conductive element. The spacing between the shielding element and the conductive element may be such as to create a suitable impedance between the shielding element and the conductive element. A “suitable impedance” may be in excess of 10MΩ/1 pF and, preferably, is about 20MΩ/2 pF.

The electrode may include a cover portion in which at least the shielding element is embedded. The cover portion may be of the same material as the carrier and may surround and envelop the shielding element and at least some of the carrier.

The electrode may be a passive device. In other words, the electrode may be free of any electronic components.

According to a second aspect of the invention, there is provided an electrode assembly which includes

an electrode as described above; and

a cable for connecting the conductive element and the shielding element of the electrode to an electronic device.

The cable may be one of a twisted wire pair and a co-axial cable. In the case of use in noisy environments or in the case of a length of cable exceeding a distance of greater than a predetermined amount, the cable may be shielded. Thus, if the cable is a twisted wire pair, the cable may include a shielding sleeve. Where the cable is a coaxial cable, the cable may be double shielded having an external shielding sleeve or screen.

The cable may include at least one active conductor and at least one secondary conductor, the at least one active conductor connecting the conductive element of the electrode to an input of the electronic device, which may, for example, be an amplifier, and the at least one secondary conductor connecting the shielding element of the electrode to shielding associated with the electronic device. If present, an external shielding sleeve of the cable may connect a secondary shield terminal of the amplifier to an on board signal ground.

According to a third aspect of the invention, there is provided a system for sensing electrophysiological signals, the system including

at least one electrode having shielding;

at least one amplifier for amplifying a signal received from the at least one electrode, the amplifier having a shielding input and the shielding of the electrode being connected to the shielded input of the at least one amplifier; and

a power supply for powering the at least one amplifier, a reference voltage derived from the power supply being used as a reference signal for the at least one electrode.

In an embodiment, the shielding of the at least one electrode may also be connected to the power supply to provide the reference voltage.

The at least one electrode may be a passive electrode, i.e. the electrode may be free of electronic components.

Where the distance between the at least one electrode and the at least one amplifier exceeds a predetermined distance, for example, about 2 m or where the system is used in a noisy environment, the cable may also be shielded.

The, or each, amplifier may be a high input impedance amplifier. By “high input impedance” is meant an input impedance exceeding about 10¹²Ω and, preferably, exceeding about 10¹³Ω.

The at least one amplifier may include a pre-amplifier stage. Input terminals of the pre-amplifier stage may be shielded.

An output of the pre-amplifier stage may be coupled to a second gain stage. The second gain stage may be a low pass filter stage. In AC, the coupling may be effected by a high pass filter and, in DC, the coupling may be effected by a conductance.

Optionally, inputs of the second gain stage may be shielded as well.

Preferably, the system is a multi-channel system including a plurality of electrodes and amplifiers, each electrode being as described above with reference to the first aspect of the invention and each electrode being connected to its associated amplifier via a cable. The pre-amplifier stages of the amplifiers may be connected together so that a shield connection of an inverting input of a preceding pre-amplifier forms a common reference electrode signal and is connected to an inverting input of a succeeding, or subsequent, pre-amplifier. Instead, the pre-amplifier stages of the amplifiers may be connected together so that a shield connection of an inverting input of a first pre-amplifier forms a common reference electrode signal and is connected to an inverting input of each subsequent pre-amplifier.

The power supply may be connected to a compensated voltage divider having a mid-point ground. A ground signal from the electrode may be coupled by a protection impedance to the mid-point ground of the power supply. The ground signal may, optionally, be connected to the mid-point ground of the voltage divider via a non-inverting input of an adder circuit.

A further reference or ground electrode may be connected to the at least one amplifier. The further reference electrode may be used where it is desired to improve the signal to noise ratio of the amplifier. Such further reference electrode may be connected via a switch, for example, a toggle switch, so that it can be used as desired.

In an embodiment, the further reference electrode may be a right leg driver (RLD) electrode, or a driven grounding electrode. The RLD electrode may be driven by a voltage follower circuit and, once again, may be used where it is desired to improve the signal to noise ratio of the amplifier.

The power supply may be mounted off the circuit board housing the at least one amplifier.

A digital conversion stage may be connected to an output of the at least one amplifier for converting analogue signals output from the amplifier to digital signals. At least one of a data communication stage and a data storage stage may be connected to an output of the digital conversion stage.

The power supply may provide power to the digital conversion stage, the data communication stage and the data storage stage.

### BRIEF DESCRIPTION OF DRAWINGS

Embodiments of the invention are now described by way of example with reference to the accompanying drawings in which:—

FIG. 1 shows a schematic, block diagram of an embodiment of a system for sensing electrophysiological signals;

FIG. 2 shows a circuit diagram of a first embodiment of an amplifier for use with the system of FIG. 1;

FIG. 3 shows a circuit diagram of a second embodiment of an amplifier for use with the system of FIG. 1;

FIG. 4 shows a circuit diagram of a third embodiment of an amplifier for use with the system of FIG. 1;

FIG. 5 shows a circuit diagram of a fourth embodiment of an amplifier for use with the system of FIG. 1;

FIG. 6 shows an embodiment of the connection of pre-amplifier stages of the amplifiers of FIGS. 2-4 for a three channel system;

FIG. 7 shows a schematic, front view of a first embodiment of an electrode for use with the system of FIG. 1;

FIG. 8 shows a schematic, rear view of the electrode of FIG. 7;

FIG. 9 shows a schematic, side view of the electrode of FIG. 7;

FIG. 10 shows a schematic, side view of a first embodiment of an electrode assembly incorporating the electrode of FIG. 7 with an insulating cover portion omitted;

FIG. 11 shows a schematic, side view of the assembly including the insulating cover portion;

FIG. 12 shows a schematic, front view of a second embodiment of an electrode for use with the system of FIG. 1;

FIG. 13 shows a schematic, rear view of the electrode of FIG. 12;

FIG. 14 shows a schematic, side view of the electrode of FIG. 12;

FIG. 15 shows a schematic, side view of a second embodiment of an electrode assembly incorporating the electrode of FIG. 12;

FIG. 16 shows a schematic, front view of a third embodiment of an electrode for use with the system of FIG. 1;

FIG. 17 shows another embodiment of the connection of pre-amplifier stages of the amplifiers of FIGS. 2-4 for a three channel system;

FIG. 18 shows a schematic, block diagram of the system for sensing electrophysiological signals including a digital communication stage;

FIG. 19 shows a schematic, block diagram of the system for sensing electrophysiological signals including a digital data storage stage; and

FIG. 20 shows a schematic, block diagram of the power supply of the system

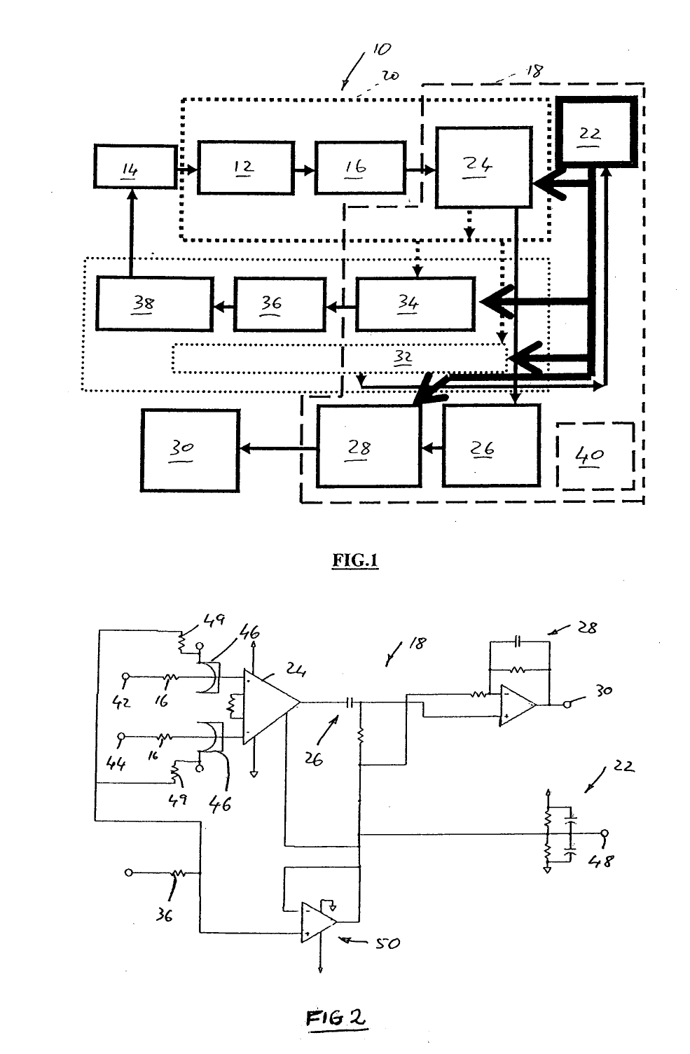

### DETAILED DESCRIPTION OF EXEMPLARY EMBODIMENTS

In the drawings, reference numeral 10 generally designates an embodiment of a system for sensing electrophysiological signals. The system 10 comprises a plurality of electrodes 12 mountable on or close to a subject's body 14. The electrodes 12 are coupled, via a coupling impedance 16, to an amplifier 18. The system 10 includes shielding 20. The shielding 20 includes shielding of each of the sensing electrodes 12, as will be described in greater detail below. Further, the system 10 includes a power supply 22 for supplying power to the amplifier 18.

The amplifier 18 includes a pre-amplifier stage, or pre-amplifier, 24. An output of the pre-amplifier 24 is connected to coupling circuitry 26, the output of the coupling circuitry 26, in turn, being connected to a high gain amplifier/low pass filter second stage 28 to provide an output signal 30 which can be further processed or displayed.

Optionally, the system 10 includes grounding circuitry and, more particularly, right leg driver (RLD) grounding suppression circuitry 32. The grounding suppression circuitry 32 includes a grounding circuit 34, an output of which is connected to a coupling impedance 36. One or more RLD grounding electrodes 38 is connected to the coupling impedance 36. The grounding electrodes 38 are associated with the subject of body 14 to provide additional grounding in situations where it is desired to improve the signal to noise ratio of the system 10.

As will be described in greater detail below, the system 10 may be used in noisy environments. In such environments, or where the electrodes 12 are spaced from its associated amplifier 18 by greater than a predetermined distance, for example, approximately 2 m, the system 10 includes a guard feature 40. The guard feature 40 is shielding of the cable connecting the electrode 12 to its associated amplifier 18 as will be described in greater detail below.

The coupling impedance 16 is, in the case of a DC coupling, a protection resistor or a capacitor in the case of an AC coupling. In the latter case, a biasing capacitive impedance may be included, one terminal of the biasing impedance being connected to the pre-amplifier 24 and the other terminal being connected to a signal ground 48. The value of the protection resistor is calculated in accordance with the following equation:

$$R = \frac{{V}_{S}}{200 n} \times {10}^{- 9}$$

where n is the number of electrode leads connected in the system 10 for multi-channel system and V_{S} is the voltage supply value from the power supply 22.

The value of the coupling capacitor according to the desired bandwidth must not exceed approximately 15 nF and must include a parallel, parasitic resistor having a resistance greater than 100 GΩ. The value of the biasing capacitive impedance must also not exceed approximately 15 nF and includes a parallel, parasitic resistor having a resistance greater than 100 GΩ. Generally, the bandwidth of the system formed by the coupling impedance and the biasing impedance must be at least the same as the bandwidth of the amplifier 18. Coupling through the capacitor transforms the system 10 into one able to be used in a contactless manner. In other words, the electrodes 12 do not need to be applied directly to the skin of the subject's body 14 but can be held in spaced relationship from, or in loose contact with, the skin of the subject's body 14 but still pick up signals from the body 14. In addition, the use of a capacitor (that has an insulator material between conductive parts) ensures that there is no physical contact between the amplifier 18 and the subject's body 14.

The amplifier 18 can adopt various forms, as will be described in greater detail below. However, each amplifier 18 has the pre-amplifier 24 which is based on a chip INA 116 (and all its variants) available from Burr-Brown TI.

The shielding 20 is the particular printed circuit board layout 18 built around the pre-amplifier chip 24 and the particular wiring to the electrode 12 associated with that amplifier 18 to minimise noise.

The power supply 22 is a circuit that furnishes a signal ground reference in the middle of a voltage source value to the amplifier 18, independent of the value of that voltage source.

The coupling circuitry 26 is an impedance that couples the pre-amplifier stage 24 to the low pass filter stage 28. In the case of a DC coupling, the coupling circuit is a resistor. In the case of an AC coupling, the coupling circuitry is a high pass filter having the required bandwidth.

The low pass filter stage 28 is a second signal gain stage and offers a low pass filtering feature which is an active low pass filter. In the preferred embodiments, the low pass filter is in the form of an OPA2336 (and all its variants) chip available from Burr-Brown TI for a low pass voltage supply applications of up to approximately 6 volts. A Burr-Brown TI chip OPA2477 (and all its variants), once again available from Burr-Brown TI, is used for high voltage applications of up to 36 volts. In the case where the active lead guard feature 40 is used, an OPA124 (and all its variants) Burr-Brown TI chip is used. In all the applications, active low pass filtering and passive high pass filtering is used.

The grounding suppression circuitry 32 comprises the grounding circuitry 34 which is the circuit that drives the feedback electrode 38 to the subject. It is a voltage follower circuit, as will be described in greater detail below with reference to FIG. 4 of the drawings and, once again, uses the OPA2336 chip from Burr-Brown TI. The circuitry 34 is connected via the coupling impedance 36 to the grounding electrode 38 which is used for RLD grounding.

Referring now to FIGS. 2-5 of the drawings, various embodiments of the amplifier 18 are illustrated and are described in greater detail.

In FIG. 2 of the drawings, a first embodiment of the amplifier 18 is shown. The amplifier 18 includes the pre-amplifier 24 having a non-inverting input 42 and an inverting input 44. Both inputs 42 and 44 are shielded by shielding 46 forming part of the shielding 20 of the system 10. The shielding 46 shielding the input 42 is independent of the shielding 46 shielding the input 44. There may, however, be implementations of the system 10 where it is desired to connect the shielding 46 of the inputs 42 and 44 to create a unique shielding arrangement. The shielding 46 includes copper traces on the printed circuit board on which components of the pre-amplifier 24 are mounted.

Signals received from a pair of electrodes 12 are fed through the coupling impedance 16 to the pre-amplifier 24. Shielding of each of the electrodes 12 is connected to the shielding 46 of each of the relevant inputs 42, 44 of the pre-amplifier 24, the shielding of the electrodes 12 forming part of the shielding 20 of the system 10.

An output signal from the pre-amplifier 24 is coupled in AC via the high pass filter 26 to the second gain/low pass filter stage 28. The low pass filter stage 28 is implemented using the Burr-Brown IT chip of the required voltage. For a supply voltage of between about 3V and 6V, an OPA2336 (and all its variants) chip is used. For a supply voltage of greater than 6V and up to 36V, an OPA2477 (and all its variants) chip is used and for a supply voltage of less than 3V, an OPA333 (and all its variants) chip is used.

The signal ground 48 is tied to the voltage supply ground of the power supply 22 in the case of a dual voltage power supply to create a middle point ground. The power supply 22 is arranged off the printed circuit board and, in the case of a multi channel system 10, is common to all the channels.

In this embodiment, the signal from each of the shielding terminals 46 is fed through a coupling impedance 49, of equal value to the coupling impedance 16, connected to the input terminals 42, 44 of the pre-amplifier 24 to a non-inverting input of an adder circuit 50. The adder circuit 50 is used to drive the signal ground 48 using voltage supply boot strapping techniques.

As indicated above, the system 10 does not require any further grounding electrodes. The grounding or reference electrode voltage value is provided by the signal ground 48 as a reference for the pre-amplifier 24. Nevertheless, a further reference electrode (not shown) can be connected to the adder circuit 50 via the coupling impedance 36. The connection of the additional reference electrode may be enabled by a toggle switch (not shown) The additional reference electrode is used in situations where it is desired to improve the signal to noise ratio of the system 10, for example, where the system 10 is used in a noisy environment.

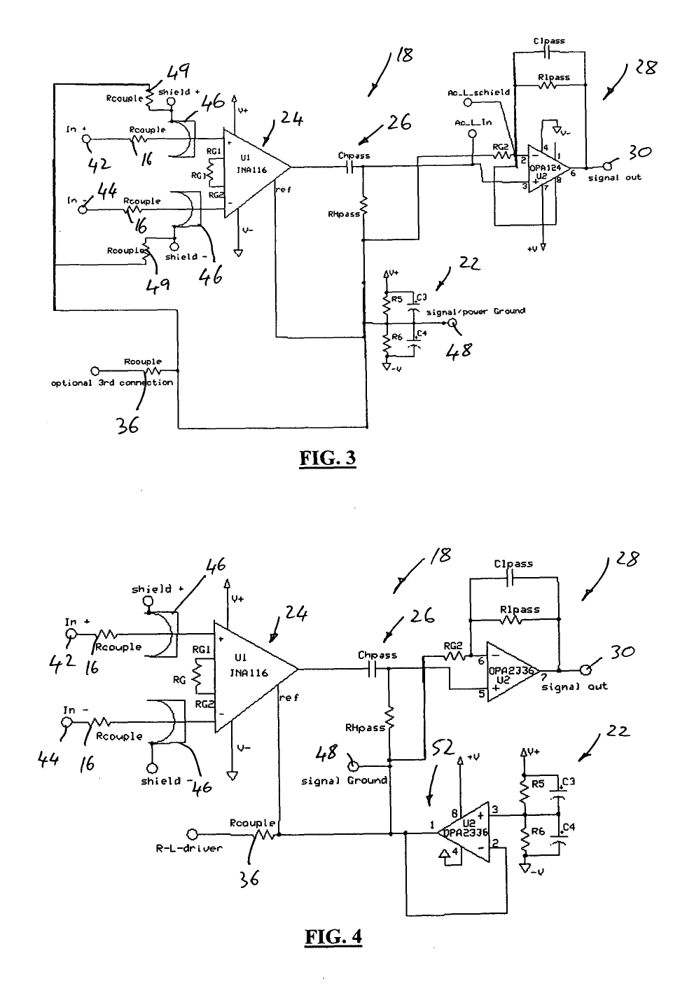

Referring now to FIG. 3 of the drawings, a second embodiment of the amplifier 18 is shown. With reference to FIG. 2 of the drawings, like reference numerals refer to like parts unless otherwise specified.

In this embodiment, the inputs to the low pass filter stage 28 are shielded by the active lead guard 40 of the system 10. Further, in this embodiment, the signal ground is obtained by a compensated resistant voltage divider forming the power supply 22. The voltage divider creates a middle point ground in case of a single voltage supply and, in the case of a dual supply, the middle voltage value can be tied to the obtained virtual ground point.

The signal coming from each shielding 46 is fed through a coupling impedance 49 of the same value as the coupling impedance 16. The signals from the shields 46 are summed in the virtual ground point.

Once again, this amplifier 18 also does not require the use of any additional grounding electrodes but, optionally, a further grounding electrode can be connected to the summing point via the coupling impedance 36. This may be effected by a toggle switch (not shown).

The low pass filter stage is implemented by way of a Burr-Brown TI OPA124 (and all its variants) chip.

Referring to FIG. 4 of the drawings, a further embodiment of the amplifier 18 is shown. Once again, with reference to the previous drawings, like reference numerals refer to like parts, unless otherwise specified.

In this embodiment, the signal ground 48 is obtained by a compensated resistive voltage divider forming the power supply 22 to create a middle point ground. In the case of a dual supply, the middle voltage value is tied to the virtual ground point. The middle point ground is insulated by a voltage follower circuit 52. The voltage follower circuit is implemented by a Burr-Brown IT OPA2336 (and all its variants) chip for a supply voltage of less than 6V, a Burr-Brown IT OPA2477 (and all its variants) chip for a supply voltage greater than 6V and up to 36V or a Burr-Brown IT OPA333 (and all its variants) chip for a supply voltage of less than 3V. The voltage follower circuit 52, as indicated above, forms part of the circuitry 34 of the RLD grounding circuitry 32. The follower circuitry 52 is used to drive the RLD grounding electrode 38 connected to the coupling impedance 36.

Once again, the use of this additional grounding electrode is optional and is used when the system is to be used in a noisy environment.

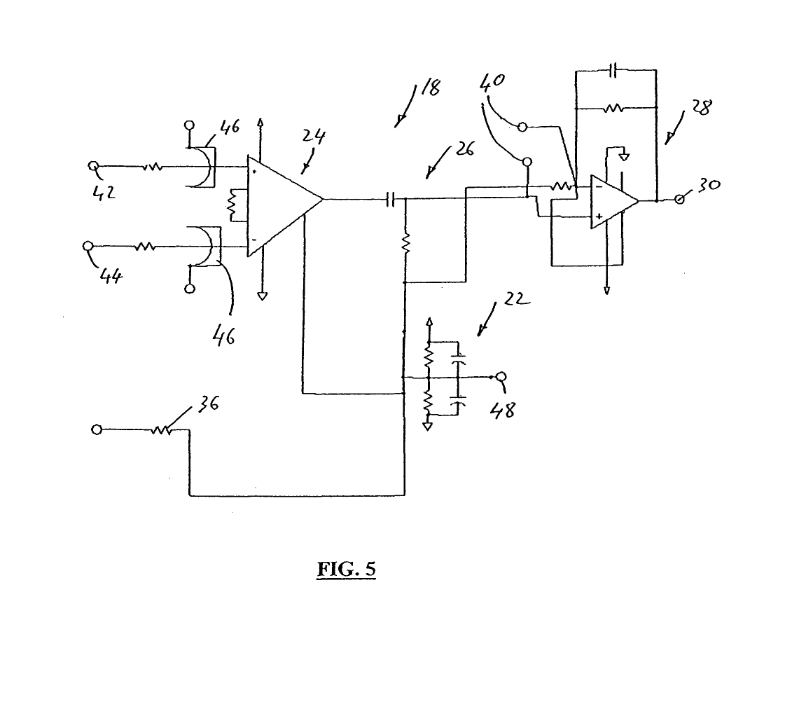

In FIG. 5 of the drawings, a further embodiment of the amplifier 18 is shown and, once again, with reference to the previous drawings, like reference numerals refer to like parts, unless otherwise specified. This embodiment is similar to the embodiment described above with reference to FIG. 3 except the signals from the shield 46 are not connected to the signal ground 48.

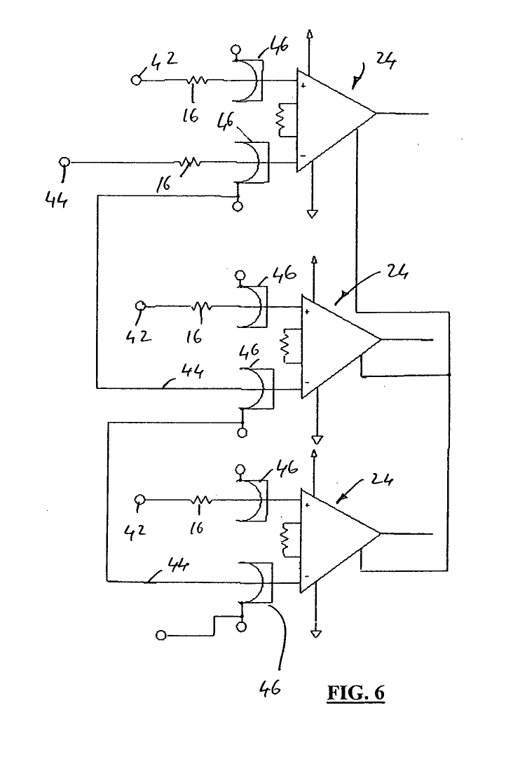

In FIG. 6 of the drawings, a multi-channel implementation is illustrated, part of the system 10 only being shown. In this embodiment, the pre-amplifiers 24 of each of the amplifiers 18 are interconnected and all reference a common reference electrode.

More particularly, as shown, the shielding 46 of the inverting input 44 of the first pre-amplifier 24 forms the common reference electrode and provides an input signal to the inverting input 44 of the second pre-amplifier 24. A signal from the shielding 46 of the inverting input 44 of the second per-amplifier 24 is used as an input signal to the inverting input 44 of the third pre-amplifier 24. In multi channel systems having more than 3 stages, the connection pattern is repeated. It is also to be noted that, instead of using the inverting input 44 of each pre-amplifier 24, the non-inverting input 42 could be used. In that case, the shielding 46 associated with the non-inverting input 42 would form the common reference electrode and provide an input signal to the non-inverting input 42 of the second pre-amplifier 24 and so on.

In FIG. 17 another embodiment of the pre-amplification stage of a multi-channel system 10 is shown. With reference to FIG. 6, like reference numerals refer to like parts unless otherwise specified. In this embodiment, the shielding 46 of the inverting input 44 of the first-preamplifier 24 forms the common reference electrode and provides an input to the inverting input 44 of each of the second and subsequent pre-amplifiers 24.

Referring now to FIGS. 7-11 of the drawings, a first embodiment of an electrode 12 for use in the system 10 is illustrated. The electrode 12 comprises a conductive element 54. The conductive element 54 is a metal plate or, instead, is of a conductive elastomeric material such as a conductive silicone. In the case of a metal plate, the metal may be selected from the group consisting of silver, gold or various silver plated or gold plated plates.

The conductive element 54 is carried by a carrier 56. The carrier 56 is of an insulating material and, more particularly, an insulating elastomeric material such as a natural or a synthetic rubber.

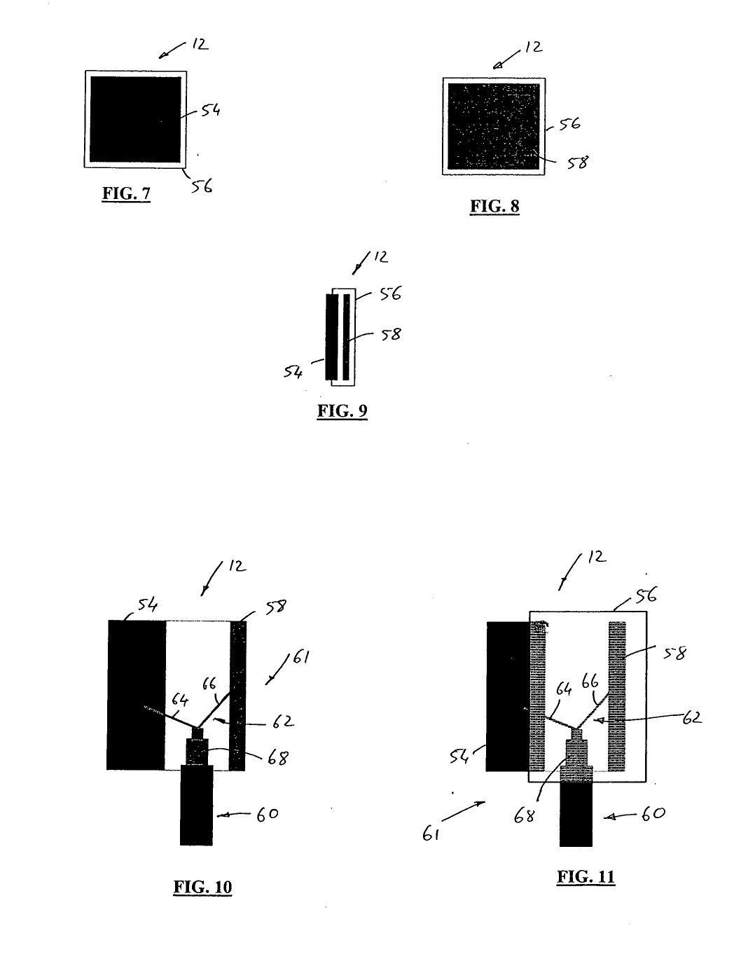

The electrode 12 further includes a shielding element 58. The shielding element 58 is, as illustrated more clearly in FIGS. 9 and 11 of the drawings, embedded in the carrier 56. The shielding plate 58 forms part of the shielding 20 of the system 10. The shielding plate 58 is spaced apart from the conductive element 54 so that a sufficiently high impedance is created between the conductive element 54 and the shielding element 58. Typically, this impedance is of the order of 20MΩ/2 pF or more.

It is to be noted that the electrode 12 is passive and carries no electronic components in it, all the components being housed in the amplifier 18, itself.

The electrode 12 is connected via a cable 60 to the input of the pre-amplifier 24 of its associated amplifier 18. The electrode 12 and the cable 60, together, form an electrode assembly 61.

The cable 60 is a stainless steel twisted pair 62. One conductor 64 of the twisted pair 62 is connected to the conductive element 54 and the other conductor 66 of the pair 62 is connected to the shielding element 58.

Where the electrode assembly 61 is to be used in a very noisy environment or over distances exceeding approximately 2 m, the cable 60 is a shielded cable having an external screen 68. The external screen 68 is used to connect to the signal ground 48 of the amplifier 18.

The conductor 64 of the cable 60 is connected to one of the inputs 42, 44 of the pre-amplifier 24. The conductor 66, or in the case of a coaxial cable, the principal screen of the coaxial cable, is connected to the relevant shielding 46 of the pre-amplifier 24.

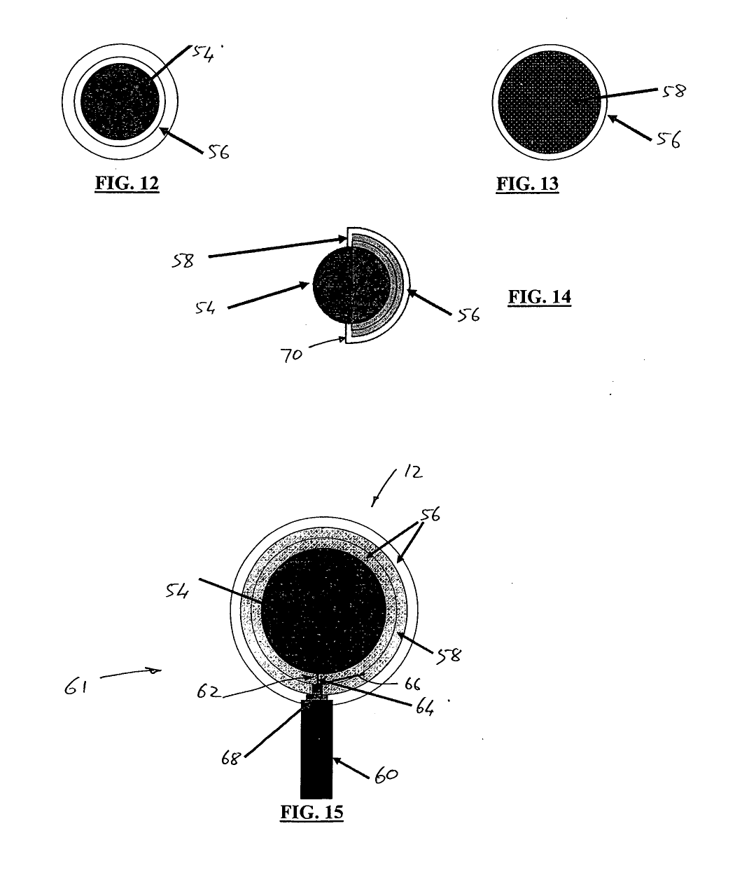

Referring now to FIGS. 12-15 of the drawings, a second embodiment of an electrode 12 and electrode assembly 61 are shown. With reference to FIGS. 7-11 of the drawings, like reference numerals refer to like parts, unless otherwise specified. This embodiment of electrode 12 is intended particularly for use with hirsute individuals, or on the head of an individual, where it is necessary to penetrate hair covering skin of the body 14 of the subject.

In this embodiment, the conductive element 54 is a spherical member which, once again, is of metal or conductive silicone. The conductive element 54 is partially embedded in the insulating carrier 56 so that approximately half of it projects beyond a surface 70 of the carrier 56 as shown in FIG. 14 of the drawings.

The shielding element 58 is semi-spherical and is embedded in the carrier 56 to surround the conductive element 54 partially.

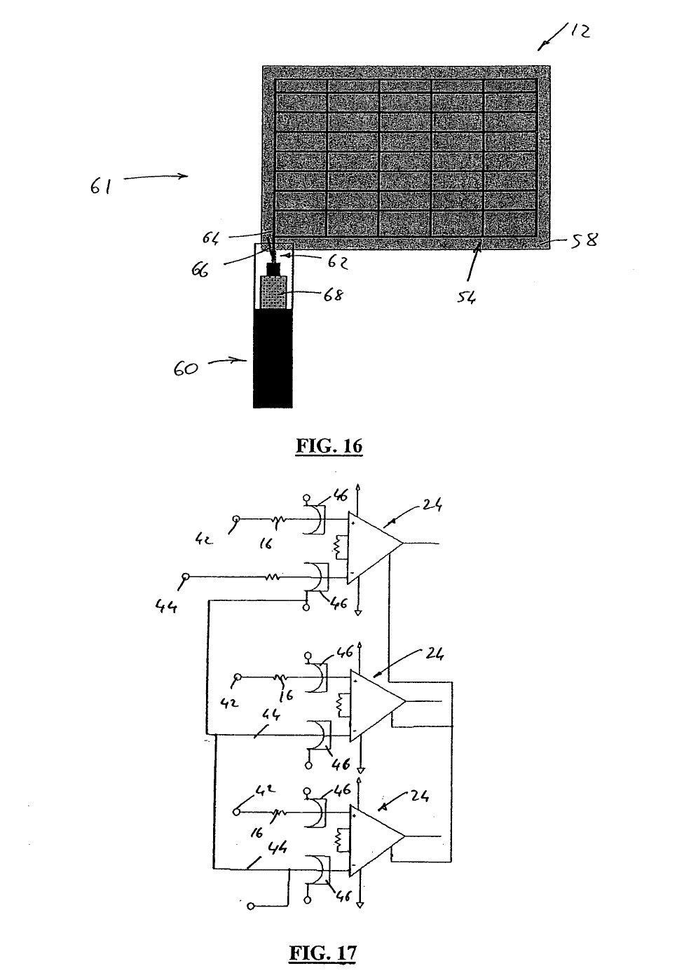

As shown in FIG. 15 of the drawings, the cable 60 is, once again, a twisted pair 62 with the active conductor 64 connected to the non-inverting input 42 of the pre-amplifier 24 and the conductor 66 connected to the inverting input 44 of the pre-amplifier 24.

In FIG. 16 of the drawings, yet a further embodiment of an electrode assembly 61 is shown. In this embodiment, the insulating carrier is omitted for the sake of clarity. The conductive element 54 is in the form of a lattice or mesh and is supported in spaced relationship relative to the shielding element 58. The cable 60 comprises a twisted pair 62 having the conductor 64 connected to the conductive element 54 and the secondary conductor 66 connected to the shielding element 58. This embodiment of electrode assembly 61 is intended particularly for use in wearing within clothing of a user and to be held in spaced relationship relative to the body 14 of the subject.

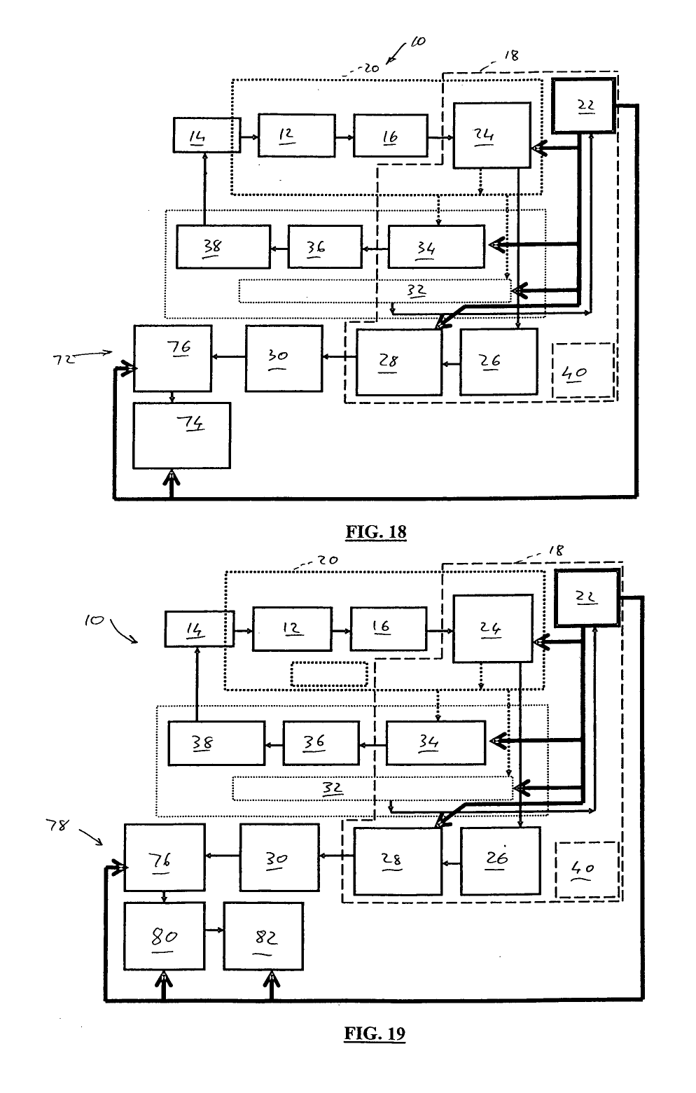

FIG. 18 of the drawings shows the system 10 connected to a data communication stage 72. The data communication stage comprises a digital data communication module 74. Because the data communication module 74 is digital, the analogue signal from the output 30 of the system 10 is first converted to a digital signal using an analogue to digital converter 76. The converter 76 includes analogue multiplexing circuitry, sample and hold circuitry and analogue to digital conversion circuitry. The data communication module 74 is any suitable device such as, for example, a USB communication device, a serial communication channel, a wireless communication channel (such as Bluetooth), or the like. Data flow from the data communication module 74 is controlled by a microcontroller chip (not shown) integrated in the data communication module 74. The power supply 22 provides power to the components of the data communication stage 72.

FIG. 19 of the drawings shows the system connected to a data storage stage 78. In this embodiment, an output from the analogue to digital converter 76 is connected to a digital data storage module 80 containing removable, digital mass storage media 82. The data storage module 80 includes a microcontroller (not shown) for controlling data flow. Once again, the power supply 22 provides power to the components of the data storage stage 78.

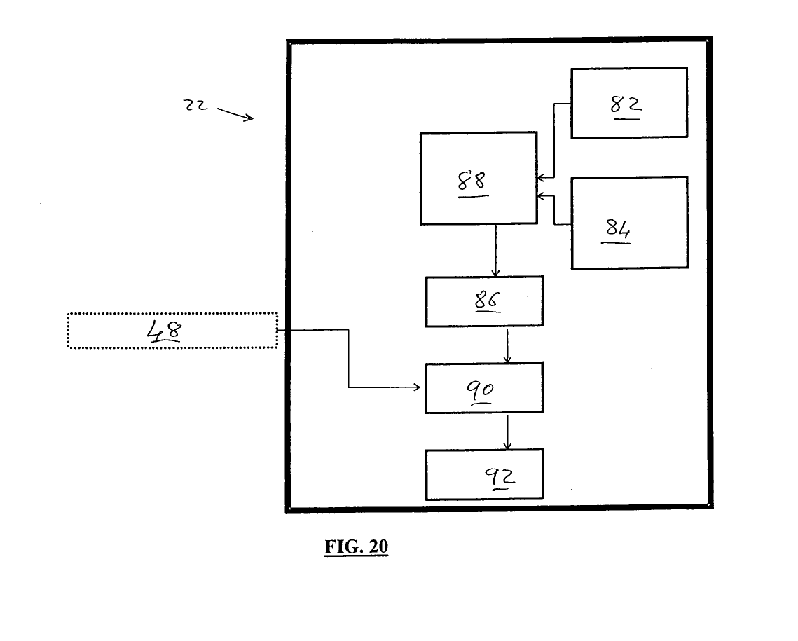

Referring now to FIG. 20 of the drawings, a block diagram of the power supply 22 is shown. The power supply 22 includes an external power source 82 and/or an internal power source 84, such as a battery pack. Examples of external power sources include USB, firewire, bus power supply lines, or any other suitable external DC power input). The power sources 82, 84 are connected to a DC-DC conversion module 86. The module 86 generates the required voltage and current values. The power sources 82, 84 are connected to the conversion module 86 through a medically approved insulating buffer, for example a 4000 Vᵣₘₛ buffer. A ground of the power supply 22 is coupled with the signal ground 48 via a coupling module 90 to obtain the required power outputs 92.

In use, the system 10 is intended for sensing electrophysiological signals such as generated when conducting an ECG, an EEG or an EMG. Thus, electrodes 12 are mounted in the desired positions on or close to the body 14 of the subject to be examined. The electrodes 12 are connected via their cables 60 to their associated amplifiers 18. Because there is no electronics within each electrode 12, the electrodes are far more lightweight. Due to the interconnection of the electrodes 12 as described above with reference to FIGS. 6 and 17 of the drawings, numerous electrodes can be used in a multi-channel configuration without degradation of the signals. Also, because of the high input impedance of the amplifiers 18, the need for a conductive gel or paste to maintain conductive contact between the conductive elements 54 of the electrodes 12 and the subject's body 14 is obviated. Thus, signals sent by the electrodes 12 are fed to the amplifiers 18 for signal processing. The use of the signal ground 48 obviates the need for a separate ground electrode. However, as indicated above, where it is desired to improve the signal to noise ratio of the system 10, an optional reference electrode can be connected via the coupling impedance 36.

It is therefore an advantage of embodiments of the invention that a system 10 is provided which does not require the use of conductive paste or gels to maintain electrical communication between the electrodes 12 and the subject's body 14. In addition, because there are no electronic components carried by the electrodes 12, the danger associated with having powered components in close proximity to the subject's body is obviated. Due to the high input impedance of the amplifiers 18 of the system 10, a contactless system 10 is able to be implemented.

It is a further advantage of embodiments of the invention that the need for a separate reference or ground electrode is obviated but one can be provided when it is desired to provide an improved signal to noise ratio to the system 10.

It is therefore an advantage of embodiments of the invention that a compact, easy to operate system 10 is provided which does not require as rigorous preparation as previous methods in mounting the electrodes in position. This lends the system for use in applications where it may be difficult to prepare a subject's body such as, for example, in the case of taking measurements of domestic animals or livestock.

It will be appreciated by persons skilled in the art that numerous variations and/or modifications may be made to the invention as shown in the specific embodiments without departing from the scope of the invention as broadly described. The present embodiments are, therefore, to be considered in all respects as illustrative and not restrictive.

1. An electrode for a system for sensing electrophysiological signals, the electrode comprising
a carrier;
a conductive element mounted on the carrier; and
a shielding element carried by the carrier and arranged in spaced relationship relative to the conductive element.

2. The electrode of claim 1 in which the carrier is of an insulating material.

3. The electrode of claim 2 in which the insulating material is a non-conducting elastomeric material.

4. The electrode of claim 1 in which the conductive element is at least one of a metal element and a conductive elastomer.

5. The electrode of claim 1 in which the shielding element has a shape complementary to its associated conductive element.

6. The electrode of claim 5 in which the shielding element is maintained by the carrier in spaced, parallel relationship to at least a part of a periphery or a surface of the conductive element.

7. The electrode of claim 6 in which the shielding element is a conductive plate shaped to complement the periphery or surface of the conductive element.

8. The electrode of claim 7 in which the spacing between the shielding element and the conductive element is such as to create a suitable impedance between the shielding element and the conductive element.

9. The electrode of claim 1 which includes a cover portion in which at least the shielding element is embedded.

10. The electrode of claim 9 in which the cover portion is of the same material as the carrier and surrounds and envelops the shielding element and at least some of the carrier.

11. The electrode of claim 1 which is a passive device.

12. An electrode assembly which includes
an electrode comprising
a carrier;
a conductive element mounted on the carrier; and
a shielding element carried by the carrier and arranged in spaced relationship relative to the conductive element; and
a cable for connecting the conductive element and the shielding element of the electrode to an electronic device.

13. The assembly of claim 12 in which the cable is one of a twisted wire pair and a co-axial cable.

14. The assembly of claim 12 in which the cable is shielded.

15. The assembly of claim 14 in which the cable includes at least one active conductor and at least one secondary conductor, the at least one active conductor connecting the conductive element of the electrode to an input of the electronic device and the at least one secondary conductor connecting the shielding element of the electrode to shielding associated with the electronic device.

16. A system for sensing electrophysiological signals, the system including
at least one electrode having shielding;
at least one amplifier for amplifying a signal received from the at least one electrode, the amplifier having a shielding input and the shielding of the electrode being connected to the shielded input of the at least one amplifier; and
a power supply for powering the at least one amplifier, a reference voltage derived from the power supply being used as a reference signal for the at least one electrode.

17. The system of claim 16 in which the shielding of the at least one electrode is also connected to the power supply to provide the reference voltage.

18. The system of claim 16 in which the at least one electrode is a passive electrode.

19. The system of claim 18 in which, where the distance between the at least one electrode and the at least one amplifier exceeds a predetermined distance, for example, about 2 m or where the system is used in a noisy environment, the cable is also shielded.

20. The system of claim 16 in which the, or each, amplifier is a high input impedance amplifier.

21. The system of claim 16 in which the at least one amplifier includes a pre-amplifier stage.

22. The system of claim 21 in which input terminals of the pre-amplifier stage are shielded.

23. The system of claim 22 in which an output of the pre-amplifier stage is coupled to a second gain stage.

24. The system of claim 23 in which, in AC, the coupling is effected by a high pass filter and, in DC, the coupling is effected by a conductance.

25. The system of claim 21 in which the system is a multi-channel system including a plurality of electrodes and amplifiers, each electrode comprising
a carrier;
a conductive element mounted on the carrier; and
a shielding element carried by the carrier and arranged in spaced relationship relative to the conductive element
and each electrode being connected to its associated amplifier via a cable.

26. The system of claim 25 in which the pre-amplifier stages of the amplifiers are connected together so that a shield connection of an inverting input of a preceding pre-amplifier forms a common reference electrode signal and is connected to an inverting input of a succeeding pre-amplifier.

27. The system of claim 25 in which the pre-amplifier stages of the amplifiers are connected together so that a shield connection of an inverting input of a first pre-amplifier forms a common reference electrode signal and is connected to an inverting input of each subsequent pre-amplifier.

28. The system of claim 16 in which the power supply is connected to a compensated voltage divider having a mid-point ground.

29. The system of claim 28 in which a ground signal from the electrode is coupled by a protection impedance to the mid-point ground of the power supply.

30. The system of claim 16 in which a further reference or ground electrode is connected to the at least one amplifier.

31. The system of claim 16 in which the power supply is mounted off the circuit board housing the at least one amplifier.

32. The system of claim 16 in which a digital conversion stage is connected to an output of the at least one amplifier for converting analogue signals output from the amplifier to digital signals.

33. The system of claim 32 in which at least one of a data communication stage and a data storage stage is connected to an output of the digital conversion stage.
